(19) United States
(12) Patent Application Publication
(10) Pub. No.: US 2022/0280478 A1
(43) Pub. Date: Sep. 8, 2022

(54) METHODS OF USING RIFAMYCIN SV FOR THE TREATMENT OF SICKLE CELL DISEASE

(71) Applicant: Bausch Health Ireland, Limited, Dublin (IR)

(72) Inventors: Ezra R. Lowe, Bridgewater, NJ (US); Zeev Heimanson, Calabasas, CA (US)

(73) Assignee: Bausch Health Ireland, Limited, Dublin (IR)

(21) Appl. No.: 17/687,534

(22) Filed: Mar. 4, 2022

Related U.S. Application Data
(60) Provisional application No. 63/157,258, filed on Mar. 5, 2021.

(51) Int. Cl. A61K 31/395; A61K 9/20; A61K 9/28; A61P 7/06

(52) U.S. Cl. CPC A61K 31/395; A61K 9/2013; A61K 9/2018; A61K 9/2009; A61K 9/2813; A61K 9/282; A61K 9/2846; A61P 7/06

(57) ABSTRACT

Provided herein is the use of rifamycin SV the treatment of sickle cell disease (SCD).

### RELATED APPLICATIONS

This application claims priority to U.S. Provisional Application No. 63/157,258, filed Mar. 5, 2021, the entire contents of which are incorporated herein by reference.

### BACKGROUND

Sickle cell disease (SCD) affects approximately 100,000 Americans and while the standard of care has improved, there remains an urgent need for new and efficacious SCD therapies.

### SUMMARY

The invention described herein meets the needs in the field by providing a treatment for sickle cell disease (SCD) through the administration of rifamycin SV to patients in need thereof.

Patients having SCD may have recurrent painful vaso-occlusive crises (VOCs), which is the most common clinical manifestation of SCD. VOC occurs when the patient's microcirculation is obstructed by sickled red blood cells (RBCs), which may result in ischemic injury, ulcers, priapism, organ damage, and spontaneous abortion. Furthermore, patients having SCD may, overall, have a poor quality of life and a shortened lifespan.

Neutrophils have been implicated in regulating VOC in SCD patients. SCD patients with WBC>15×10⁹/L are more likely to develop stroke, acute chest syndrome, and premature death. Neutrophils in SCD patients are also shown to exhibit increased levels of activation molecules, including CD64 and CD11b/CD18, with their sera having elevated levels of soluble CD62L. A subset of neutrophils known as circulating aged neutrophils (CANs) are substantially elevated. CANs are characterized by having a high surface expression of CXCR4 and low CD62L. Activated and aged neutrophils may be immobilized in the circulatory system on the endothelium and form the nidus for the adhesion of sickled RBCs, which may lead to VOC.

It has been reported that modulating intestinal microbial composition may be a therapeutic option in treating SCD patients to reduce VOC through the reduction of activated and aged neutrophils.

Rifamycin SV is a broad spectrum, semi-synthetic, orally non-absorbable antibiotic which can be used for the treatment of bacterial infections of the colon such as traveler's diarrhea and infectious colitis. More recently, alternative delivery forms of rifamycin SV have been developed to improve local delivery. One such form relies on the application of MMX® technology, which comprises a multi-matricial core composition surrounded by a pH-sensitive coating, allowing the tablets to arrive unaltered to the terminal part of the ileum, where they start to gradually release rifamycin SV.

Accordingly, rifamycin SV and its delivery forms (e.g., rifamycin SV MMX®) described herein provide a therapy for treating SCD in a patient by, for example, and without being limited to any one theory of the invention, (1) reducing levels of elevated circulating aged neutrophils (CANs), and/or (2) reducing or preventing the occurrence of vaso-occlusive crises (VOCs).

In an embodiment, the invention described herein includes a method of treating sickle cell disease (SCD) in a patient in need thereof comprising administering at least one rifamycin SV composition to the patient. In an embodiment, the rifamycin SV compositions described herein are in solid dosage form. In an embodiment, the rifamycin SV compositions described herein are oral compositions. In an embodiment, the rifamycin SV compositions described herein are immediate release, delayed release, or both extended and delayed release compositions. In an embodiment, the rifamycin SV compositions described herein are formulated at a dose of 600 mg of rifamycin SV. In an embodiment, the rifamycin SV compositions described herein are formulated to release the rifamycin SV substantially in the small intestine. In some embodiments, the at least one rifamycin SV composition comprises rifamycin SV MMX® as described e.g., Luigi Moro et al. Antibiotics 2021, 10, 167 and WO 2019/092614, the entirety of which are incorporated herein by reference.

In some embodiments, the method of treating sickle cell disease (SCD) comprises reducing elevated levels of circulating aged neutrophils (CANs) in the patient. In some embodiments, the method of treating sickle cell disease (SCD) in a patient comprises treating vaso-occlusive crisis (VOC) in the patient. In some embodiments, treating vaso-occlusive crisis (VOC) in the patient comprises (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC.

### DETAILED DESCRIPTION

Provided herein is the use of rifamycin SV for the treatment of Sickle Cell Disease (SCD).

“Rifamycin SV” and “rifamycin” are used interchangeably and are known in the art as the chemical compound having the Chemical Abstracts No. 6998-60-3.

Pharmaceutically acceptable salts of rifamycin SV are also contemplated for use in the disclosed compositions and methods of use.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein the term “treatment of sickle cell disease (SCD)” may refer to the amelioration, prevention, or reduction in frequency of one or more symptoms of SCD. For example, “treatment of sickle cell disease (SCD)” may refer to the reduction of elevated levels of circulating aged neutrophils (CANs) in a patient, where such levels of CANs are elevated as compared, for example, to levels of CANs that would be expected in a patient who is not diagnosed as having SCD. As another example, the “treatment of sickle cell disease (SCD)” may refer to treating vaso-occlusive crisis (VOC) in the patient, where “treating vaso-occlusive crisis (VOC) or “treating vaso-occlusive crises (VOCs),” as the case may be, may refer to (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC.

In an embodiment, the invention described herein includes the use of at least one rifamycin SV composition (e.g., rifamycin SV MMX®) for the treatment of sickle cell disease (SCD).

In an embodiment, the rifamycin SV compositions described herein are in solid dosage form. In an embodiment, the rifamycin SV compositions described herein are oral compositions. In an embodiment, the rifamycin SV compositions described herein are immediate release, delayed release, or both extended and delayed release compositions. In an embodiment, the rifamycin SV compositions described herein are formulated at a dose of 1 mg to 1000 mg of rifamycin SV. In an embodiment, the rifamycin SV compositions described herein are formulated to release the rifamycin SV substantially in the small intestine. In some embodiments, the at least one rifamycin SV composition comprises rifamycin SV MMX® as described e.g., Luigi Moro et al. Antibiotics 2021, 10, 167 and WO 2019/092614. In an embodiment, the at least one rifamycin SV composition is formulated to release the rifamycin SV substantially following passage of the solid dosage form into the pylorus passage in the proximal part of the intestine.

In an embodiment, the at least one rifamycin SV composition further comprises one or more of a lipophilic compound, a hydrophilic compound, and an amphiphilic compound.

In an embodiment, the at least one rifamycin SV composition further comprises a gastro-resistant coating.

In an embodiment, the at least one rifamycin SV composition is in solid dosage form and is released from the solid dosage form when the solid dosage form is placed in an aqueous medium having a pH in the range of about pH 5 to about pH 7.5.

In an embodiment, the at least one rifamycin SV composition comprises a core and a gastro-resistant coating covering the core. In one aspect, the core is a matrix, mixture, compressed blend or a dispersion of the ingredients found in the core. In one aspect the coating is applied directly on the core. In one embodiment, the core may also comprise one or more of microcrystalline cellulose, lactose (such as lactose monohydrate), colloidal silicon dioxide, povidone and/or co-povidone, and microcrystalline cellulose.

In an embodiment, the at least one rifamycin SV composition, when administered to the subject, produces a t_{max,0-24} of the rifamycin SV in the plasma of the subject of about 9.50 hours.

In an embodiment, the at least one rifamycin SV composition, when administered to the subject, produces a C_{max,0-6} of the rifamycin SV in the plasma of the subject of about 2.30±2.14 ng/mL.

In an embodiment, the at least one rifamycin SV composition, when administered to the subject, produces a C_{max,0-24} of the rifamycin SV in the plasma of the subject of about 6.23±4.52 ng/mL.

In an embodiment, the at least one rifamycin SV composition, when administered to the subject, produces an AUC₀₋₆ of the rifamycin SV in the plasma of the subject of about 3.34±2.96 (ng)(h)/mL.

In an embodiment, the at least one rifamycin SV composition, when administered to the subject, produces an AUC₀₋₂₄ of the rifamycin SV in the plasma of the subject of about 947.92±20.24 (ng)(h)/ml.

In an embodiment, the at least one rifamycin SV composition, when administered to the subject, comprises a urine elimination rate of the rifamycin SV in a subject to which the composition is administered of not more than 1% of the total amount of rifamycin SV administered to the subject.

Other suitable dosage forms that can be used with the disclosed include, but are not limited to, capsules, tablets, mini-tablets, beads, beadlets, pellets, granules, granulates, and powder. Suitable dosage forms may be coated, for example using an enteric coating. In some embodiments, the compositions are formulated as tablets, caplets, or capsules. In one embodiment, the compositions are formulated as a tablet.

Provided compositions may be formulated such that a dosage of between 0.001-100 mg/kg body weight/day of rifamycin SV can be administered to a patient receiving these compositions. It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated.

In some embodiments, the method of treating sickle cell disease (SCD) comprises reducing elevated levels of circulating aged neutrophils (CANs) in the patient. In some embodiments, the method of treating sickle cell disease (SCD) in a patient comprises treating vaso-occlusive crisis (VOC) in the patient.

In an embodiment, the invention includes a method of treating sickle cell disease (SCD) in a patient in need thereof by administering a dose of a rifamycin SV composition to the patient, wherein the dose comprises about 1 mg to about 1000 mg of rifamycin SV or a salt thereof, or about 50 mg to about 750 mg of rifamycin SV or a salt thereof, or about 250 mg to about 700 mg of rifamycin SV or a salt thereof, or about 450 mg to about 650 mg of rifamycin SV or a salt thereof, or about 550 mg to about 650 mg of rifamycin SV or a salt thereof.

In an embodiment, the invention includes a method of treating sickle cell disease (SCD) in a patient in need thereof by administering a dose of a rifamycin SV composition to the patient, wherein the dose comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, or 800 mg of rifamycin SV or a salt thereof.

In an embodiment, the invention includes a method of treating sickle cell disease (SCD) in a patient in need thereof by administering a dose of a rifamycin SV composition to the patient, wherein the dose comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, or 800 mg or rifamycin SV or a salt thereof.

In an embodiment, the invention includes a method of treating sickle cell disease (SCD) in a patient in need thereof by administering a dose of a rifamycin SV composition to the patient, wherein the dose comprises at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, or 800 mg of rifamycin SV or a salt thereof.

In an embodiment, the invention includes a method of treating sickle cell disease (SCD) in a patient in need thereof by administering a dose of a rifamycin SV composition to the patient, wherein the dose comprises about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, or 700 mg of rifamycin SV or a salt thereof.

In an embodiment, the invention includes a method of treating sickle cell disease (SCD) in a patient in need thereof by administering a dose of a rifamycin SV composition to the patient, wherein the dose comprises at least 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, or 700 mg of rifamycin SV or a salt thereof.

In an embodiment, the invention includes a method of treating sickle cell disease (SCD) in a patient in need thereof by administering a dose of a rifamycin SV composition to the patient, wherein the dose comprises at most 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, or 700 mg of rifamycin SV or a salt thereof.

In an embodiment, the invention includes a method of treating sickle cell disease (SCD) in a patient in need thereof by administering a dose of a rifamycin SV composition to the patient, wherein the dose comprises about 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, or 625 mg of rifamycin SV or a salt thereof.

In an embodiment, the invention includes a method of treating sickle cell disease (SCD) in a patient in need thereof by administering a dose of a rifamycin SV composition to the patient, wherein the dose comprises at least 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, or 625 mg of rifamycin SV or a salt thereof.

In an embodiment, the invention includes a method of treating sickle cell disease (SCD) in a patient in need thereof by administering a dose of a rifamycin SV composition to the patient, wherein the dose comprises at most 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, or 625 mg of rifamycin SV or a salt thereof.

In some embodiments of the foregoing methods, the rifamycin SV composition comprises about 600 mg of rifamycin SV or a salt thereof.

In some embodiments of the foregoing methods, the rifamycin SV composition comprises a sodium salt of rifamycin SV.

In some embodiments of the foregoing methods, the rifamycin SV composition comprises one or more excipients selected from the group consisting of ascorbic acid, lecithin, stearic acid, sodium carboxymethyl cellulose, mannitol, colloidal anhydrous silica, and magnesium stearate.

In some embodiments of the foregoing methods, the rifamycin SV composition is in tablet form comprising a tablet core, wherein the tablet core comprises:

(a) rifamycin SV or a salt thereof in an amount of about 45% to about 55% by weight of the tablet core;

(b) ascorbic acid in an amount of about 2% to about 3% by weight of the tablet core;

(c) lecithin in an amount of about 0.5% to about 1.5% by weight of the tablet core;

(d) stearic acid in an amount of about 2% to about 3% by weight of the tablet core;

(e) sodium carboxymethyl cellulose in an amount of about 10% to about 20% by weight of the tablet core;

(f) mannitol in an amount of about 15% to about 25% by weight of the tablet core;

(g) colloidal anhydrous silica in an amount of about 1% to about 2% by weight of the tablet core; and

(h) magnesium stearate in an amount of about 1.5% to about 3% by weight of the tablet core.

In some embodiments of the foregoing methods, the rifamycin SV composition is in tablet form comprising a tablet core, wherein the tablet core comprises:

(a) rifamycin SV sodium in an amount of about 45% to about 55% by weight of the tablet core;

(b) ascorbic acid in an amount of about 2% to about 3% by weight of the tablet core;

(c) lecithin in an amount of about 0.5% to about 1.5% by weight of the tablet core;

(d) stearic acid in an amount of about 2% to about 3% by weight of the tablet core;

(e) sodium carboxymethyl cellulose in an amount of about 10% to about 20% by weight of the tablet core;

(f) mannitol in an amount of about 15% to about 25% by weight of the tablet core;

(g) colloidal anhydrous silica in an amount of about 1% to about 2% by weight of the tablet core; and

(h) magnesium stearate in an amount of about 1.5% to about 3% by weight of the tablet core.

In some embodiments of the foregoing methods, the rifamycin SV composition is in tablet form comprising a tablet core, wherein the tablet core comprises:

(a) rifamycin SV or a salt thereof in an amount of about 53.1% by weight of the tablet core;

(b) ascorbic acid in an amount of about 2.7% by weight of the tablet core;

(c) lecithin in an amount of about 0.9% by weight of the tablet core;

(d) stearic acid in an amount of about 2.7% by weight of the tablet core;

(e) sodium carboxymethyl cellulose in an amount of about 17.7% by weight of the tablet core;

(f) mannitol in an amount of about 19.0% by weight of the tablet core;

(g) colloidal anhydrous silica in an amount of about 1.8% by weight of the tablet core; and

(h) magnesium stearate in an amount of about 2.2% by weight of the tablet core.

In some embodiments of the foregoing methods, the rifamycin SV composition is in tablet form comprising a tablet core, wherein the tablet core comprises:

(a) rifamycin SV sodium in an amount of about 53.1% by weight of the tablet core;

(b) ascorbic acid in an amount of about 2.7% by weight of the tablet core;

(c) lecithin in an amount of about 0.9% by weight of the tablet core;

(d) stearic acid in an amount of about 2.7% by weight of the tablet core;

(e) sodium carboxymethyl cellulose in an amount of about 17.7% by weight of the tablet core;

(f) mannitol in an amount of about 19.0% by weight of the tablet core;

(g) colloidal anhydrous silica in an amount of about 1.8% by weight of the tablet core; and

(h) magnesium stearate in an amount of about 2.2% by weight of the tablet core.

In some embodiments of the foregoing methods, the rifamycin SV composition comprises a coating, which may be applied to the tablet core, wherein the coating may comprise one or more excipients selected from the group consisting of methacrylic acid, methyl methacrylate, methacrylic acid-methyl methacrylate copolymer, titanium dioxide, red ferric oxide, talc, triethylcitrate, and polyethylene glycol.

In some embodiments of the foregoing methods, the rifamycin SV composition comprises a first coating, which may be applied to the tablet core, wherein the coating comprises one or more excipients selected from the group consisting of methacrylic acid, methyl methacrylate, methacrylic acid-methyl methacrylate copolymer, titanium dioxide, red ferric oxide, talc, and triethylcitrate. In some embodiments, the rifamycin SV composition comprises a second coating, which may be applied to the first coating, wherein the coating comprises polyethylene glycol (e.g., polyethylene glycol 6000).

In some embodiments of the foregoing methods, the rifamycin SV composition is in tablet form comprising a tablet core and a tablet coating, wherein:

(a) the tablet core comprises rifamycin SV or a salt thereof, a hydrophilic substance, a lipophilic substance, and an amphiphilic substance; and

(b) the coating comprises a gastro-resistant substance as described in U.S. Pat. No. 8,263,120, the entirety of which is incorporated herein by reference.

In some embodiments of the foregoing methods, a lipophilic substance is a substance having a melting point lower than 90° C., such as, for example, beeswax, carnauba wax, stearic acid, stearin and the like; an amphiphilic substance is represented by substances selected, for example, from phospholipids, ceramides, sphingomyelins, lecithins, alkyl block copolymers, salts of sulphated alkyl acids, polyoxyethylenated alkyl, derivatives of sorbitan and the like; and a hydrophilic substance is represented by generally cross-linked or linear polymeric or copolymeric substances, which are known as hydrogels, that is to say, substances capable of increasing their mass and their weight, owing to the polar groups present in the main or side polymer chains, when they come into contact with molecules of water, including, without limitation, cellulose derivatives, such as hydroxyalkylcelluloses, alkylcelluloses, carboxyalkylcelluloses and their salts or derivatives, polyvinyl alcohols, carboxyvinyl derivatives, polysaccharide derivatives of anionic or cationic nature, such as, for example, hyduronic acid, glucuronic acid, or glucosamines, pectins and/or their derivatives.

In some embodiments of the foregoing methods, a gastro-resistant substance may comprise polymers of acrylic and methacrylic acids (Eudragit) and/or derivatives of cellulose phthalate.

In some embodiments of the foregoing methods, the dose of the rifamycin SV composition may be administered to the patient once a day (QD), twice a day (BID), three times a day (TID), or four times a day (QID).

In some embodiments of the foregoing methods, the dose of the rifamycin SV composition may be administered to the patient BID or TID.

In some embodiments of the foregoing methods, the dose of the rifamycin SV composition may be administered to the patient once a day (QD), twice a day (BID), three times a day (TID), or four times a day (QID), for a period of at least 1, 2, 3, 4, 5, 6, 7, 8, 9, 10, 11, 12, 13, or 14 days.

In some embodiments of the foregoing methods, the dose of the rifamycin SV composition may be administered to the patient once a day (QD), twice a day (BID), three times a day (TID), or four times a day (QID), for a period of at most 1, 2, 3, 4, 5, 6, 7, 8, 9, 10, 11, 12, 13, or 14 days.

In some embodiments of the foregoing methods, the dose of the rifamycin SV composition may be administered to the patient once a day (QD), twice a day (BID), three times a day (TID), or four times a day (QID), for a period of about 1, 2, 3, 4, 5, 6, 7, 8, 9, 10, 11, 12, 13, or 14 days.

In some embodiments of the foregoing methods, the dose of the rifamycin SV composition may be administered to the patient BID or TID for a period of at least 1, 2, 3, 4, 5, 6, 7, 8, 9, 10, 11, 12, 13, or 14 days.

In some embodiments of the foregoing methods, the dose of the rifamycin SV composition may be administered to the patient BID or TID for a period of at most 1, 2, 3, 4, 5, 6, 7, 8, 9, 10, 11, 12, 13, or 14 days.

In some embodiments of the foregoing methods, the dose of the rifamycin SV composition may be administered to the patient BID or TID for a period of about 1, 2, 3, 4, 5, 6, 7, 8, 9, 10, 11, 12, 13, or 14 days.

In some embodiments of the foregoing methods, the rifamycin SV composition may comprise one or more tablets of a composition described in U.S. Pat. No. 8,263,120 or U.S. Patent Application Publication No. 2020/0281850, the entirety of which are incorporated herein by reference.

### EXAMPLES

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Example 1: Proposed QD Treatment of Sickle Cell Disease

A treatment is proposed in patients having sickle cell disease in order to reduce the VOCs in said patients by treating them once per day with a 600 mg dose of rifamycin SV according to Table 1, which may be prepared according to the methods of U.S. Patent Application Publication No. 2020/0281850.

TABLE 1
Extended Release Coated Rifamycin SV Tablet

| | | Amount per tablet |
|---|---|---|
| | Ingredient | (mg) |
| Tablet Core | Rifamycin SV Sodium | 600.0 |
| | Ascorbic Acid | 30.0 |
| | Lecithin | 10.0 |
| | Stearic Acid | 30.0 |
| | Sodium Carboxymethyl Cellulose | 200.0 |
| | Mannitol | 215.0 |
| | Colloidal Anhydrous Silica | 20.0 |
| | Magnesium Stearate | 25.0 |
| Tablet Coating | Eudragit ® L30 D55 | 49.8 |
| | Titanium Dioxide | 2.7 |
| | Red Ferric Oxide | 0.2 |
| | Talc | 22.2 |
| | Triethylcitrate | 5.1 |

SCD patients may be treated once per day with the tablet of Table 1 for a period of six months to determine whether therapy has the benefit of reducing VOCs in the patient.

### Example 2: Proposed BID Treatment of Sickle Cell Disease

A treatment is proposed in patients having sickle cell disease in order to reduce the VOCs in said patients by treating them BID with a 600 mg dose of rifamycin SV according to Table 1, which may be prepared according to the methods of U.S. Patent Application Publication No. 2020/0281850.

SCD patients may be treated BID with the tablet of Table 1 for a period of six months to determine whether therapy has the benefit of reducing VOCs in the patient.

### Example 3: Proposed QD Treatment of Sickle Cell Disease

A treatment is proposed in patients having sickle cell disease in order to reduce the VOCs in said patients by treating them once per day with 194 mg of rifamycin (AEMCOLO (rifamycin) delayed-release tablets).

SCD patients may be treated once per day with AEMCOLO (rifamycin) delayed-release tablets for a period of six months to determine whether therapy has the benefit of reducing VOCs in the patient.

### Example 4: Proposed BID Treatment of Sickle Cell Disease

A treatment is proposed in patients having sickle cell disease in order to reduce the VOCs in said patients by treating them BID with 194 mg of rifamycin (AEMCOLO (rifamycin) delayed-release tablets).

SCD patients may be treated BID with AEMCOLO (rifamycin) delayed-release tablets for a period of six months to determine whether therapy has the benefit of reducing VOCs in the patient.

1. A method of treating sickle cell disease (SCD) in a patient in need thereof comprising administering at least one rifamycin SV composition to the patient.

2. A method of reducing elevated levels of circulating aged neutrophils (CANs) in a patient in need thereof comprising administering at least one rifamycin SV composition to the patient.

3. A method of treating vaso-occlusive crises (VOCs) in a patient in need thereof comprising administering at least one rifamycin SV composition to the patient.

4. The method according to claim 1, wherein the at least one rifamycin SV composition comprises a solid dosage form.

5. The method according to claim 1, wherein the at least one rifamycin SV composition is an oral composition.

6. The method according to claim 1, wherein the at least one rifamycin SV composition is formulated for extended release, delayed release, or both extended and delayed release.

7. The method according to claim 1, wherein the at least one rifamycin SV composition is formulated to release substantially in the small intestine.

8. The method according to claim 1, wherein the at least one rifamycin SV composition is formulated to release the rifamycin SV substantially following passage of the solid dosage form into the pylorus passage in the proximal part of the intestine.

9. The method according to claim 1, wherein the at least one rifamycin SV composition further comprises one or more of a lipophilic compound, a hydrophilic compound, and an amphiphilic compound.

10. The method according to claim 1, wherein the at least one rifamycin SV composition further comprises a gastro-resistant coating.

11. The method according to claim 1, wherein the at least one rifamycin SV composition is released from the solid dosage form when the solid dosage form is placed in an aqueous medium having a pH in the range of about pH 5 to about pH 7.5.

12. The method according to claim 1, wherein the solid dosage form comprises a core and a gastro-resistant coating covering the core.

13. The method according to claim 1, wherein administration of the at least one rifamycin SV composition produces a t_{max,0-24} of the rifamycin SV in the plasma of the subject of about 9.50 hours.

14. The method according to claim 1, wherein administration of the at least one rifamycin SV composition produces a C_{max,0-6} of the rifamycin SV in the plasma of the subject of about 2.30±2.14 ng/mL.

15. The method according to claim 1, wherein administration of the at least one rifamycin SV composition produces a C_{max,0-24} of the rifamycin SV in the plasma of the subject of about 6.23±4.52 ng/mL.

16. The method according to claim 1, wherein administration of the at least one rifamycin SV composition produces an AUC₀₋₆ of the rifamycin SV in the plasma of the subject of about 3.34±2.96 (ng)(h)/mL.

17. The method according to claim 1, wherein administration of the at least one rifamycin SV composition produces an AUC₀₋₂₄ of the rifamycin SV in the plasma of the subject of about 947.92±20.24 (ng)(h)/ml.

18. The method according to claim 1, wherein administration comprise a urine elimination rate of the rifamycin SV in a subject to which the composition is administered of not more than 1% of the total amount of rifamycin SV administered to the subject.

19. (canceled)
